(19) United States
(12) Patent Application Publication
(10) Pub. No.: US 2006/0241122 A1
(43) Pub. Date: Oct. 26, 2006

(54) Combination therapy for the treatment of neoplasms

(76) Inventors: Margaret S. Lee, Middleton, MA (US); M. James Nichols, Boston, MA (US); Amy Beth Wilson, Cambridge, MA (US); Grant R. Zimmermann, Somerville, MA (US)

Correspondence Address: CLARK & ELBING LLP, 101 FEDERAL STREET, BOSTON, MA, 02110, US

(21) Appl. No.: 10/559,039

(22) PCT Filed: May 27, 2004
(86) PCT No.: PCT/US04/16653

§ 371 Date: May 8, 2006

Related U.S. Application Data
(60) Provisional application No. 60/474,704, filed on May 30, 2003.

(51) Int. Cl. A61K 31/496; A61K 31/4196; A61K 31/4178; A61K 31/401; A61K 31/366; A61K 31/22

(52) U.S. Cl. 514/254.07; 514/383; 514/397; 514/423; 514/460; 514/548

(57) ABSTRACT

The invention features compositions, methods, and kits for the treatment of neoplasms.

### BACKGROUND OF THE INVENTION

The invention relates to the treatment of neoplasms such as cancer.

Cancer is a disease marked by the uncontrolled growth of abnormal cells. Cancer cells have overcome the barriers imposed on normal cells, which have a finite lifespan, to grow indefinitely. As the growth of cancer cells continue, genetic alterations may persist until the cancerous cell has manifested itself to pursue a more aggressive growth phenotype. If left untreated, metastasis, the spread of cancer cells to distant areas of the body by way of the lymph system or bloodstream, may ensue, destroying healthy tissue.

According to a recent American Cancer Society study, approximately 1,268,000 new cancer cases were expected to be diagnosed in the United States in the year 2001 alone. Lung cancer is the most common cancer-related cause of death among men and women, accounting for over 28% of all cancer-related deaths. It is the second most commonly occurring cancer among men and women; it has been estimated that there were more than 169,000 new cases of lung cancer in the U.S. in the year 2001 and accounting for 13% of all new cancer diagnoses. While the rate of lung cancer cases is declining among men in the U.S., it continues to increase among women. According to the American Cancer Society, an estimated 157,400 Americans were expected to die due to lung cancer in 2001.

Cancers that begin in the lungs are divided into two major types, non-small cell lung cancer and small cell lung cancer, depending on how the cells appear under a microscope. Non-small cell lung cancer (squamous cell carcinoma, adenocarcinoma, and large cell carcinoma) generally spreads to other organs more slowly than does small cell lung cancer. Small cell lung cancer is the less common type, accounting for about 20% of all lung cancer.

Other cancers include brain cancer, breast cancer, cervical cancer, colon cancer, gastric cancer, kidney cancer, leukemia, liver cancer, lymphoma, ovarian cancer, pancreatic cancer, prostate cancer, rectal cancer, sarcoma, skin cancer, testicular cancer, and uterine cancer. These cancers, like lung cancer, are sometimes treated with chemotherapy.

Antiproliferative agents currently in use or in clinical trials include paclitaxel, docetaxel, tamoxifen, vinorelbine, gemcitabine, cisplatin, etoposide, topotecan, irinotecan, anastrozole, rituximab, trastuzumab, fludarabine, cyclophosphamide, gentuzumab, carboplatin, interferon, and doxorubicin. The most commonly used anticancer agent is paclitaxel, which is used alone or in combination with other chemotherapy drugs such as: 5-fluorouracil, doxorubicin, vinorelbine, cytoxan, and cisplatin.

### SUMMARY OF THE INVENTION

We have discovered that the combination of an azole and an HMG-CoA reductase inhibitor is more effective in reducing the growth of neoplastic cells than either agent alone. Thus, these agents, as well as their structural or functional analogs, can be used in an antineoplastic combination of the invention.

Accordingly, in one aspect, the invention features a method for treating a patient who has a neoplasm (e.g., cancer) or is at risk for developing a neoplasm by administering to the patient an HMG-CoA reductase inhibitor and an azole, wherein the compounds are administered simultaneously or within 28 days of each other in amounts sufficient to inhibit or prevent the growth of the neoplasm. Suitable HMG-CoA reductase inhibitors include, but are not limited to simvastatin, lovastatin, mevastatin, pravastatin, monacolin M, monacolin X, fluvastatin, atorvastatin, cerivastatin, rosuvastatin, fluindostatin, velostatin, compactin, dihydrocompactin, rivastatin, dalvastatin, and pitavastatin. Suitable azoles include, but are not limited to, fluconazole, itraconazole, hydroxyitraconazole, posaconazole, saperconazole, ketoconazole, clotrimazole, terconazole, econazole, tioconazole, oxiconazole, butoconazole, and miconazole.

In particular embodiments of the foregoing method, one or both of the administered compounds are approved by a national pharmaceutical regulatory agency, such as the United States Food and Drug Administration (USFDA), for administration to a human. Desirably, the compounds are administered within 14 or 7 days of each other, within 24 hours of each other, within one hour of each other, or simultaneously. Most desirably, the compounds are administered in the same pharmaceutical formulation. It may be preferable to administer each compound individually, either by the same or different route of administration. Each compound may, independently, be administered by intravenous, intramuscular, subcutaneous, rectal, oral, topical, intravaginal, ophthalmic, and inhalation administration.

An HMG-CoA reductase inhibitor is desirably administered in an amount between 0.1 and 100 mg/day, more desirably between 0.1 and 50 mg/day, and most desirably between 0.1 and 5 mg/day. An azole is administered in an amount, frequency, and duration, which measurably enhances the effectiveness of an HMG-CoA reductase inhibitor; this typically is in an amount between 0.1 and 400 mg/day, 0.1 and 200 mg/day, or 0.1 and 100 mg/day. The two compounds are desirably administered in a ratio of azole to HMG-CoA reductase inhibitor of about 4:1 to about 20:1. An azole and/or an HMG-CoA reductase inhibitor can alternatively be administered as a 0.5% to 25% w/v topical formulation. Such topical formulations are particularly useful for treating cancers of the skin and glands of the dermis and epidermis (i.e., sweat glands and sebaceous glands).

The compounds can be provided together in a pharmaceutical composition that contains a pharmaceutically acceptable carrier, excipient, or diluent. Compounds employed in the methods of the invention can be provided as components of a kit. Such a kit would typically also include instructions for using the compounds in the methods of the invention. In these kits, compounds can be formulated together or separately and in individual dosage amounts.

The invention also features a method for treating a patient having cancer in which the foregoing method is performed in combination with an additional treatment for cancer, such as surgery, radiation therapy, chemotherapy, immunotherapy, anti-angiogenesis therapy, or gene therapy. The two treatments are typically within six months of each other, and may be performed concurrently. Preferably, the additional treatment is chemotherapy. Most preferably, the additional treatment includes administering to a patient cisplatin, daunorubicin, doxorubicin, etoposide, methotrexate, mercaptopurine, 5-fluorouracil, hydroxyurea, vinblastine, vincristine, paclitaxel, or any combination thereof.

Cancers that can be treated according to the methods of the invention include leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma. Preferably, the cancer being treated is lung cancer, especially lung cancer attributed to squamous cell carcinoma, adenocarcinoma, or large cell carcinoma, colorectal cancer, ovarian cancer, especially ovarian adenocarcinoma, or prostate cancer.

In particular embodiments of this invention, an azole is administered in combination with a HMG-CoA reductase inhibitor and one, two, three, or more antiproliferative agents, in amounts and frequencies sufficient to inhibit growth of the neoplasm. Typically, each is administered at least once during a 28-day period, and may, independently, be administered twice, three times, four times, or even daily (28 times) during a 28-day period, as required to inhibit growth of the neoplasm.

The invention also features a method for identifying combinations of compounds useful for treating or preventing a neoplasm in a patient in need of such treatment. The method includes (a) contacting cells in vitro with (i) an azole or an HMG-CoA reductase inhibitor and (ii) a candidate compound; and (b) determining whether the combination of the azole or HMG-CoA reductase inhibitor and the candidate compound reduces cell proliferation relative to cells contacted with the azole or HMG-CoA reductase inhibitor but not contacted with the candidate compound or cells contacted with the candidate compound but not with the azole or HMG-CoA reductase inhibitor. A reduction in cell proliferation identifies the combination as one that is useful for treating a patient in need of such treatment.

By “cancer” or “neoplasm” or “neoplastic cells” is meant a collection of cells multiplying in an abnormal manner. Cancer growth is uncontrolled and progressive, and occurs under conditions that would not elicit, or would cause cessation of, multiplication of normal cells.

By an “HMG-CoA reductase inhibitor” is a compound that inhibits the enzymatic activity of 3-hydroxy-3-methylglutaryl-coenzyme A (HMG-CoA) reductase by at least about 10%. HMG-CoA reductase inhibitors include but are not limited to simvastatin, lovastatin, mevastatin, pravastatin, monacolin M, monacolin X, fluvastatin, atorvastatin, cerivastatin, rosuvastatin, fluindostatin, velostatin, compactin, dihydrocompactin, rivastatin, dalvastatin, and pitavastatin, as well as pharmaceutically acceptable salts thereof (e.g., simvastatin sodium, lovastatin sodium, fluvastatin sodium, etc.).

By “azole” is meant any member of the class of antifungal compounds having a five-membered ring of three carbon atoms and two nitrogen atoms (e.g., imidazoles) or two carbon atoms and three nitrogen atoms (e.g., triazoles), which are capable of inhibiting fungal growth. A compound is considered “antifungal” if it inhibits growth of a species of fungus in vitro by at least 25%. Typically, azoles are administered in dosages of greater than 200 mg per day when used as an antifungal agent. Examples of azoles for use in the methods and compositions of the invention are described herein.

By an “antiproliferative agent” is meant a compound that, individually, inhibits the growth of a neoplasm. Antiproliferative agents include, but are not limited to microtubule inhibitors, topoisomerase inhibitors, platins, alkylating agents, and anti-metabolites. Particular antiproliferative agents include paclitaxel, gemcitabine, doxorubicin, vinblastine, etoposide, 5-fluorouracil, carboplatin, altretamine, aminoglutethimide, amsacrine, anastrozole, azacitidine, bleomycin, busulfan, carmustine, chlorambucil, 2-chlorodeoxyadenosine, cisplatin, colchicine, cyclophosphamide, cytarabine, cytoxan, dacarbazine, dactinomycin, daunorubicin, docetaxel, estramustine phosphate, floxuridine, fludarabine, gentuzumab, hexamethylmelamine, hydroxyurea, ifosfamide, imatinib, interferon, irinotecan, lomustine, mechlorethamine, melphalen, 6-mercaptopurine, methotrexate, mitomycin, mitotane, mitoxantrone, pentostatin, procarbazine, rituximab, streptozocin, tamoxifen, temozolomide, teniposide, 6-thioguanine, topotecan, trastuzumab, vincristine, vindesine, and vinorelbine.

By “inhibits the growth of a neoplasm” is meant measurably slows, stops, or reverses the growth rate of the neoplasm or neoplastic cells in vitro or in vivo. Desirably, a slowing of the growth rate is by at least 20%, 30%, 50%, or even 70%, as determined using a suitable assay for determination of cell growth rates (e.g., a cell growth assay described herein). Typically, a reversal of growth rate is accomplished by initiating or accelerating necrotic or apoptotic mechanisms of cell death in the neoplastic cells, resulting in a shrinkage of the neoplasm.

By “an amount that is effective to treat a neoplasm” is meant an amount of a compound, alone or in a combination according to the invention, required to inhibit the growth of a neoplasm in vivo. The effective amount of active compound(s) used to practice the present invention for therapeutic treatment of a neoplasm (e.g., cancer) varies depending upon the manner of administration, the age, body weight, and general health of the subject. Ultimately, the attending physician or veterinarian will decide the appropriate amount and dosage regimen. Such amount is referred to as an “effective” amount.

The combination of a compound of an azole with an HMG-CoA reductase inhibitor for the treatment of neoplasms allows for the administration of lower doses of each compound, providing similar efficacy or increased efficacy, compared to administration of either compound alone. The methods also allow for the administration of standard doses of each compound, providing improved efficacy, compared to the administration of either compound alone.

By a “low dosage” is meant at least 10% less than the lowest standard recommended dosage of an HMG-CoA reductase inhibitor or azole. By a “high dosage” is meant at least 5% more than the highest standard dosage of an HMG-CoA reductase inhibitor or azole. By a “moderate dosage” is meant the dosage between the low dosage and the high dosage.

By “treating” is meant administering or prescribing a pharmaceutical composition for the treatment or prevention of an inflammatory disease.

By “patient” is meant any animal (e.g., a human).

Compounds useful in the invention include those described herein in any of their pharmaceutically acceptable forms, including isomers such as diastereomers and enantiomers, salts, solvates, and polymorphs, thereof, as well as racemic mixtures and pure isomers of the compounds described herein.

Other features and advantages of the invention will be apparent from the following detailed description, and from the claims.

### DETAILED DESCRIPTION

We have discovered that azoles enhance the antiproliferative activity of HMG-CoA reductase inhibitors against cancer cells in vitro. Thus, an azole is useful in combination with an HMG-CoA reductase inhibitor for the treatment of cancer and other neoplasms. This discovery provides for combination therapies useful for the treatment of cancer and other neoplasms. The HMG-CoA reductase inhibitor/azole combination therapy may be administered with conventional pharmaceuticals useful for the treatment of cancer. Lower doses of one or more compounds may be administered when both an HMG-CoA reductase inhibitor and an azole are administered.

Based on known properties that are shared among HMG-CoA reductase inhibitors, and among azoles, structurally or functionally related compounds can be substituted in the anti-neoplastic combinations of the invention.

Information regarding each of the compounds and its analogs and metabolites is provided below.

EMG-CoA Reductase Inhibitors

HMG-CoA reductase inhibitors include simvastatin, lovastatin, mevastatin, pravastatin, monacolin M, monacolin X, fluvastatin, atorvastatin, cerivastatin, rosuvastatin, fluindostatin, velostatin, compactin, dihydrocompactin, rivastatin, dalvastatin, and pitavastatin. Typically, for treatment of high cholesterol, dosage of an HMG-CoA reductase inhibitor varies according to a patient's condition, but standard recommended dosages are as follows: simvastatin—5-80 mg/day; lovastatin—10-80 mg/day; fluvastatin—20-40 mg/day; atorvastatin—10-80 mg/day; cerivastatin—0.2-0.4 mg; pravastatin—10-80 mg/day; rosuvastatin—5-80 mg/day; and pitavastatin—2-4 mg/day.

Additional HMG-CoA reductase inhibitors and analogs thereof useful in the methods and compositions of the present invention are described in U.S. Pat. Nos. 3,983,140; 4,231,938; 4,282,155; 4,293,496; 4,294,926; 4,319,039; 4,343,814; 4,346,227; 4,351,844; 4,361,515; 4,376,863; 4,444,784; 4,448,784; 4,448,979; 4,450,171; 4,503,072; 4,517,373; 4,661,483; 4,668,699; 4,681,893; 4,719,229; 4,738,982; 4,739,073; 4,766,145; 4,782,084; 4,804,770; 4,841,074; 4,847,306; 4,857,546; 4,857,547; 4,940,727; 4,946,864; 5,001,148; 5,006,530; 5,075,311; 5,112,857; 5,116,870; 5,120,848; 5,166,364; 5,173,487; 5,177,080; 5,273,995; 5,276,021; 5,369,123; 5,385,932; 5,502,199; 5,763,414; 5,877,208; and 6,541,511; and U.S. Patent Application Publication Nos. 2002/0013334 A1; 2002/0028826 A1; 2002/0061901 A1; and 2002/0094977 A1.

Azoles

Azoles that can be employed in the methods and compositions of the invention include fluconazole, itraconazole, hydroxyitraconazole, posaconazole, saperconazole, ketoconazole, clotrimazole, terconazole, econazole, tioconazole, oxiconazole, butoconazole, and miconazole. Typically, for treatment of a fungal infection, an azole is administered in an amount according to a patient's condition, but standard recommended dosages are provided below.

Fluconazole is typically administered as a single oral dose of 150 mg to treat vaginal yeast infections. For other type of fungal infections, a standard recommended dose of between 100 and 400 mg/day is administered orally or by injection. The standard recommended dosage of itraconazole is between 100 and 400 mg/day. Administration may be by capsule, injection, or oral solution. Ketoconazole is administered as an oral suspension or in tablet form at a standard recommended dosage of between 200 and 400 mg/day.

Additional azoles and analogs thereof useful in the methods and compositions of the present invention are described in U.S. Pat. Nos. 3,575,999; 3,705,172; 3,717,655; 3,936,470; 4,062,966; 4,078,071; 4,107,314; 4,124,767; 4,144,346; 4,223,036; 4,229,581; 4,232,034; 4,244,964; 4,248,881; 4,267,179; 4,272,545; 4,307,105; 4,335,125; 4,360,526; 4,368,200; 4,402,968; 4,404,216; 4,416,682; 4,458,079; 4,466,974; 4,483,865; 4,490,530; 4,490,540; 4,503,055; 4,510,148; 4,554,286; 4,619,931; 4,625,036; 4,628,104; 4,632,933; 4,661,602; 4,684,392; 4,735,942; 4,761,483; 4,771,065; 4,789,587; 4,818,758; 4,833,141; 4,877,878; 4,916,134; 4,921,870; 4,960,782; 4,992,454; and 5,661,151.

Formulation of Pharmaceutical Compositions

Suitable modes of administration include oral, rectal, intravenous, intramuscular, subcutaneous, inhalation, topical or transdermal, vaginal, intraperitoneal (IP), intra-articular, and ophthalmic.

Administration of a of each compound of the combination may be any suitable means that results in a concentration of the compound that, combined with the other component, is anti-neoplastic upon reaching the target region. Compounds are admixed with a suitable carrier substance, and are generally present in an amount of 1-95% by weight of the total weight of the composition. The composition may be provided in a dosage form that is suitable for oral, parenteral (e.g., intravenous, intramuscular, subcutaneous), rectal, transdermal, nasal, vaginal, inhalant, or ocular administration. Thus, the composition may be in form of, e.g., tablets, capsules, pills, powders, granulates, suspensions, emulsions, solutions, gels including hydrogels, pastes, ointments, creams, plasters, drenches, delivery devices, suppositories, enemas, injectables, implants, sprays, or aerosols. The pharmaceutical compositions may be formulated according to conventional pharmaceutical practice (see, e.g., Remington: The Science and Practice of Pharmacy, (20th ed.) ed. A. R. Gennaro, 2000, Lippincott Williams & Wilkins, Philadelphia, Pa. and Encyclopedia of Pharmaceutical Technology, eds. J. Swarbrick and J. C. Boylan, 1988-2002, Marcel Dekker, New York).

Therapy

The combinations of compounds of the invention are useful for the treatment of neoplasms. Combination therapy may be performed alone or in conjunction with another therapy (e.g., surgery, radiation, chemotherapy, biologic therapy). Additionally, a person having a greater risk of developing a neoplasm (e.g., one who is genetically predisposed or one who previously had a neoplasm) may receive prophylactic treatment to inhibit or delay neoplastic formation. The duration of the combination therapy depends on the type of disease or disorder being treated, the age and condition of the patient, the stage and type of the patient's disease, and how the patient responds to the treatment.

Combination therapy may be provided wherever chemotherapy is performed: at home, the doctor's office, a clinic, a hospital's outpatient department, or a hospital. Treatment generally begins at a hospital so that the doctor can observe the therapy's effects closely and make any adjustments that are needed. The duration of the combination therapy depends on the kind of cancer being treated, the age and condition of the patient, the stage and type of the patient's disease, and how the patient's body responds to the treatment. Drug administration may be performed at different intervals (e.g., daily, weekly, or monthly) and the administration of each agent can be determined individually. Combination therapy may be given in on-and-off cycles that include rest periods so that the patient's body has a chance to build healthy new cells and regain its strength.

Depending on the type of cancer and its stage of development, the combination therapy can be used to treat cancer, to slow the spreading of the cancer, to slow the cancer's growth, to kill or arrest cancer cells that may have spread to other parts of the body from the original tumor, to relieve symptoms caused by the cancer, or to prevent cancer in the first place. Combination therapy can also help people live more comfortably by eliminating cancer cells that cause pain or discomfort.

The dosage, frequency and mode of administration of each component of the combination can be controlled independently. For example, one compound may be administered topically three times per day, while the second compound may be administered orally once per day. Combination therapy may be given in on-and-off cycles that include rest periods so that the patient's body has a chance to recovery from any as yet unforeseen side-effects. The compounds may also be formulated together such that one administration delivers both compounds.

Examples of cancers and other neoplasms include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma).

Dosages

The dosage of each compound of the claimed combinations depends on several factors, including: the administration method, the neoplasm to be treated, the severity of the neoplasm, whether the neoplasm is to be treated or prevented, and the age, weight, and health of the patient to be treated.

A compound of the combination may be administered orally in the form of tablets, capsules, elixirs or syrups, or rectally in the form of suppositories. Parenteral administration of a compound is suitably performed, for example, in the form of saline solutions or with the compound incorporated into liposomes. In cases where the compound in itself is not sufficiently soluble to be dissolved, a solubilizer such as ethanol can be applied. One skilled in the art will recognize that if an alternative compound is substituted for either a bupivacaine analog or dibucaine analog or any one of the antiproliferative agents, the correct dosage can be determined by examining the efficacy of the compound in cell proliferation assays. For topical administration, an azole or HMG-CoA reductase inhibitor is usually provided in a 0.1%-25% w/v solution, cream, or gel. The azole and HMG-CoA reductase inhibitor is usually given by the same routes of administration that are known to be effective for delivering such a compound. When used in combination therapy according to the methods of this invention, the azole or HMG-CoA reductase inhibitor is dosed in an amount and frequency equivalent to or less than those that result in effective anticancer monotherapy using that compound.

### EXAMPLE 1

### Antiproliferative Activity of Simvastatin and Itraconazole Against Non-Small Cell Lung Carcinoma A549 Cells

Inhibition of proliferation was measured by anti-proliferation assay as described below after incubation with the test compound(s) for 72 hours. The effects of varying concentrations of simvastatin, itraconazole, or a combination of simvastatin and itraconazole were compared to control wells (seeded with A549 cells, but not incubated with either simvastatin or itraconazole).

The results of this experiment are shown in Table 1. The effects of the agents alone and in combination are shown as percent inhibition of cell proliferation.

The data demonstrate that, in the present assay, simvastatin maximally inhibits cell proliferation by 95.4% at concentrations of 48 μM. The addition of 7 μM itraconazole reduces the simvastatin concentration required for nearly maximal inhibition to 12 μM, a 4-fold reduction in the concentration of simvastatin.

TABLE 1
Percent inhibition of Alamar Blue Metabolism in A549 cells

| | | Itraconazole (μM) | | | | | | | | |
|---|---|---|---|---|---|---|---|---|---|---|
| | | 0 | 0.22 | 0.44 | 0.87 | 1.7 | 3.5 | 7 | 14 | 28 |
| Simvastatin (μM) | 0 | 8.1 | 18.1 | 12.5 | 13.5 | 7.6 | 10.1 | 4.0 | 13.3 | 13.9 |
| | 0.37 | 9.9 | 16.7 | 16.4 | 14.1 | 23.0 | 23.8 | 34.8 | 32.8 | 28.4 |
| | 0.75 | 18.2 | 17.0 | 23.4 | 27.5 | 17.9 | 28.2 | 34.7 | 35.3 | 33.1 |
| | 1.5 | 25.0 | 33.6 | 26.6 | 24.1 | 29.3 | 36.5 | 40.8 | 41.7 | 38.2 |
| | 3 | 11.2 | 25.0 | 19.3 | 15.3 | 14.1 | 23.5 | 43.1 | 41.4 | 41.6 |
| | 6 | 37.0 | 44.2 | 44.4 | 20.5 | 22.8 | 51.5 | 68.1 | 71.2 | 71.4 |
| | 12 | 70.5 | 74.3 | 72.2 | 42.5 | 49.4 | 86.2 | 91.3 | 92.3 | 91.6 |
| | 24 | 89.3 | 90.0 | 89.0 | 86.9 | 89.4 | 93.0 | 94.3 | 91.3 | 93.4 |
| | 48 | 95.4 | 95.2 | 95.0 | 94.7 | 95.1 | 95.4 | 95.4 | 95.5 | 95.3 |

### EXAMPLE 2

### Antiproliferative Activity of Simvastatin and Itraconazole Against HCT116 Colorectal Carcinoma Cells

Table 2 shows the results from an anti-proliferation assay using HCT116 cells treated with simvastatin, itraconazole, or a combination of simvastatin and itraconazole. In the present assay, simvastatin shows maximal inhibition of 81.2% at 48 μM. In the presence of 7 μM itraconazole and 12 μM simvastatin, the efficacy of simvastatin is enhanced, exhibiting 92.7% inhibition, exceeding the maximal inhibition of simvastatin alone, with a four-fold reduction in concentration.

TABLE 2
Percent inhibition of Alamar Blue Metabolism in HCT116 cells

| | | Itraconazole (μM) | | | | | | | | |
|---|---|---|---|---|---|---|---|---|---|---|
| | | 0 | 0.22 | 0.44 | 0.87 | 1.7 | 3.5 | 7 | 14 | 28 |
| Simvastatin (μM) | 0 | −3.8 | −3.1 | −2.2 | 3.9 | 7.5 | 17.2 | 18.9 | 19.2 | 18.6 |
| | 0.37 | 0.4 | 0.2 | 2.6 | 6.3 | 10.9 | 24.0 | 33.2 | 28.4 | 31.6 |
| | 0.75 | 9.5 | 8.7 | 14.7 | 15.2 | 21.5 | 44.7 | 47.9 | 47.6 | 49.1 |
| | 1.5 | 28.5 | 21.2 | 23.9 | 19.7 | 38.5 | 64.0 | 72.4 | 74.2 | 68.5 |
| | 3 | 25.0 | 24.0 | 32.0 | 32.1 | 59.0 | 79.6 | 85.4 | 84.1 | 85.3 |
| | 6 | 40.0 | 52.1 | 50.7 | 63.3 | 79.1 | 89.3 | 90.2 | 90.7 | 90.0 |
| | 12 | 60.8 | 60.8 | 71.4 | 82.3 | 88.8 | 91.6 | 92.7 | 92.7 | 92.8 |
| | 24 | 74.7 | 78.2 | 83.7 | 89.4 | 91.4 | 93.3 | 93.8 | 93.8 | 93.8 |
| | 48 | 81.2 | 82.1 | 83.2 | 87.1 | 91.1 | 92.4 | 94.1 | 94.1 | 94.3 |

### EXAMPLE 3

### Antiproliferative Activity of Simvastatin and Itraconazole Against Human HCT116 Human Colorectal Carcinoma Cells

The results from a 2-fold dilution series of simvastatin sodium (Calbiochem) and itraconazole combination on HCT116 cell growth are shown in Table 3. In the present assay, simvastatin exhibits maximal inhibition of 69.8% at 22 μM. In the presence of 7 μM itraconazole and 5.5 μM simvastatin, the combination exhibits 89.9% inhibition, exceeding the maximal inhibition of simvastatin alone, with a four-fold reduction simvastatin.

TABLE 3
Percent inhibition of Alamar Blue Metabolism in HCT116 cells

| | | Itraconazole (μM) | | | | | | | | |
|---|---|---|---|---|---|---|---|---|---|---|
| | | 0 | 0.22 | 0.44 | 0.87 | 1.7 | 3.5 | 7 | 14 | 28 |
| Simvastatin (μM) | 0 | −14.4 | −12.5 | −14.1 | −10.7 | −5.9 | −2.3 | 12.2 | 10.6 | 17.2 |
| | 0.17 | −11.4 | −14.0 | −11.2 | −7.0 | −4.7 | 11.8 | 18.4 | 18.1 | 18.9 |
| | 0.34 | −13.4 | −11.3 | −10.9 | −11.1 | −6.1 | 6.3 | 19.4 | 22.1 | 25.0 |
| | 0.68 | −5.5 | −10.3 | −6.0 | −4.7 | 5.7 | 16.1 | 35.4 | 36.3 | 42.6 |
| | 1.4 | 31.3 | 9.6 | 7.5 | 20.1 | 8.3 | 41.0 | 68.8 | 69.7 | 70.4 |
| | 2.7 | 34.6 | 38.3 | 33.3 | 13.8 | 37.4 | 70.2 | 86.2 | 84.7 | 85.3 |
| | 5.5 | 35.1 | 23.9 | 43.8 | 53.2 | 65.9 | 83.1 | 89.9 | 89.4 | 88.9 |
| | 11 | 55.8 | 60.6 | 55.2 | 72.5 | 86.1 | 91.4 | 92.0 | 92.4 | 92.1 |
| | 22 | 69.8 | 65.8 | 72.5 | 85.7 | 90.3 | 92.3 | 93.3 | 92.8 | 93.3 |

### EXAMPLE 4

### Antiproliferative Activity of Simvastatin and Itraconazole Against PANC1 Human Pancreatic Carcinoma Cells

The results from a 2-fold dilution series of simvastatin and itraconazole combination on PANC1 cell growth are shown in Table 4. In the present assay, simvastatin exhibits maximal inhibition of 43.3% at 48 μM. The addition of 7 μM itraconazole reduces the simvastatin concentration required for nearly maximal inhibition to 12 μM, a four-fold reduction in the concentration of simvastatin.

TABLE 4
Percent inhibition of Alamar Blue Metabolism in PANC1 cells

| | | Itraconazole (μM) | | | | | | | | |
|---|---|---|---|---|---|---|---|---|---|---|
| | | 0 | 0.22 | 0.44 | 0.87 | 1.7 | 3.5 | 7 | 14 | 28 |
| Simvastatin (μM) | 0 | −6.7 | −5.2 | −0.1 | 2.6 | 1.9 | 1.8 | −2.6 | 2.1 | 1.7 |
| | 0.37 | 5.9 | −0.8 | −4.6 | 7.5 | 8.0 | 7.5 | 9.6 | 9.2 | 15.4 |
| | 0.75 | 5.4 | 20.0 | 19.0 | 16.5 | 11.4 | 14.1 | 22.7 | 12.3 | 15.8 |
| | 1.5 | 19.3 | 21.3 | 11.1 | 24.6 | 13.0 | 32.0 | 35.8 | 43.7 | 33.8 |
| | 3 | 6.7 | 18.0 | 16.9 | 28.5 | 32.1 | 44.8 | 53.2 | 48.5 | 52.4 |
| | 6 | 16.2 | 24.9 | 34.5 | 38.5 | 44.9 | 55.3 | 56.5 | 60.9 | 56.2 |
| | 12 | 25.0 | 24.4 | 35.8 | 44.6 | 54.0 | 59.4 | 61.0 | 61.0 | 62.0 |
| | 24 | 33.7 | 40.3 | 43.1 | 54.9 | 60.5 | 60.7 | 60.3 | 63.9 | 62.6 |
| | 48 | 43.3 | 44.3 | 54.9 | 57.7 | 61.9 | 60.5 | 62.8 | 62.9 | 63.5 |

### EXAMPLE 5

### Antiproliferative Activity of Simvastatin and Itraconazole Against SKMEL-28 Human Melanoma Cells

The results from a 2-fold dilution series of simvastatin and itraconazole combination on SKMEL-28 cell growth are shown in Table 5. In the present assay, simvastatin exhibits maximal inhibition of 40.7% at 48 μM. In the presence of 7 μM itraconazole and 6 μM simvastatin, the combination exceeds maximum inhibition, reaching 58.5% inhibition, with an 8-fold reduction in simvastatin.

TABLE 5
Percent inhibition of Alamar Blue Metabolism in SKMEL-28 cells

| | | Itraconazole (μM) | | | | | | | | |
|---|---|---|---|---|---|---|---|---|---|---|
| | | 0 | 0.22 | 0.44 | 0.87 | 1.7 | 3.5 | 7 | 14 | 28 |
| Simvastatin (μM) | 0 | −1.4 | −0.3 | 9.3 | 1.0 | 4.2 | 13.9 | 13.5 | 19.3 | 16.7 |
| | 0.37 | 17.0 | 17.0 | 3.4 | 16.8 | 11.9 | 32.6 | 33.9 | 32.7 | 48.9 |
| | 0.75 | 14.7 | 17.4 | 20.6 | 31.9 | 37.4 | 61.3 | 55.6 | 61.6 | 56.1 |
| | 1.5 | 36.6 | 27.6 | 42.5 | 31.4 | 64.1 | 63.6 | 69.2 | 71.5 | 71.7 |
| | 3 | 11.0 | 12.2 | 16.3 | 34.3 | 42.9 | 56.2 | 59.0 | 58.8 | 60.5 |
| | 6 | 20.0 | 22.8 | 27.4 | 43.1 | 54.2 | 62.4 | 58.5 | 59.4 | 61.4 |
| | 12 | 30.3 | 33.2 | 37.3 | 42.7 | 53.0 | 58.4 | 57.8 | 59.7 | 58.6 |
| | 24 | 39.7 | 40.2 | 39.0 | 45.5 | 51.2 | 57.5 | 57.2 | 57.3 | 58.1 |
| | 48 | 40.7 | 41.1 | 42.2 | 42.4 | 45.5 | 52.6 | 56.6 | 56.8 | 57.4 |

### EXAMPLE 6

### Antiproliferative Activity of Atorvastatin and Itraconazole Against DU145 Human Prostate Cancer Cells

The results from a 2-fold dilution series of an atorvastatin and itraconazole combination on DU145 cell growth are shown in Table 6. In the present assay, atorvastatin exhibits maximal inhibition of 25.6% at 36 μM. In the presence of 28 μM itraconazole, the efficacy of atorvastatin is enhanced, exhibiting 55.6% inhibition.

TABLE 6
Percent inhibition of Alamar Blue Metabolism in DU145 cells

| | | Itraconazole (μM) | | | | | | | | |
|---|---|---|---|---|---|---|---|---|---|---|
| | | 0 | 0.22 | 0.44 | 0.87 | 1.7 | 3.5 | 7 | 14 | 28 |
| Atorvastatin (μM) | 0 | −3.7 | 1.9 | 0.4 | 0.8 | −0.3 | 3.8 | −3.5 | −4.2 | −8.8 |
| | 0.28 | −3.7 | 1.4 | −0.7 | 7.6 | 8.5 | −3.7 | 1.1 | 14.1 | 4.9 |
| | 0.56 | 2.9 | 12.0 | 0.7 | 13.3 | 3.4 | 11.7 | 3.8 | 8.4 | 11.0 |
| | 1.1 | 8.0 | 15.4 | 15.9 | 19.1 | 15.3 | 15.2 | 21.1 | 8.2 | 15.6 |
| | 2.2 | 0.5 | −0.3 | 4.9 | 5.6 | 2.3 | 0.5 | 2.2 | 4.4 | 5.5 |
| | 4.5 | −0.8 | −0.4 | 3.4 | 9.7 | 6.6 | 3.3 | 10.6 | 6.3 | 5.2 |
| | 9 | −0.3 | 0.1 | 3.0 | 5.2 | 4.9 | 5.9 | 10.1 | 7.8 | 8.4 |
| | 18 | 2.2 | 3.4 | 6.3 | 11.7 | 8.0 | 19.1 | 29.1 | 25.7 | 27.7 |
| | 36 | 25.6 | 24.5 | 31.2 | 28.1 | 39.8 | 40.3 | 53.2 | 52.9 | 55.6 |

### EXAMPLE 7

### Antiproliferative Activity of Atorvastatin and Itraconazole Against HCT116 Human Colorectal Carcinoma Cells

The results from a 2-fold dilution series of atorvastatin and itraconazole, either alone or in combination, on HCT116 cell growth are shown in Table 7. In the present assay, atorvastatin exhibits maximal inhibition of 69.0% at 36 μM. In the presence of 7 μM itraconazole and 9 μM atorvastatin, the combination exhibits 82.9% inhibition, exceeding the maximal inhibition of atorvastatin with only one fourth the amount of atorvastatin.

TABLE 7
Percent inhibition of Alamar Blue Metabolism in HCT116 cells

| | | Itraconazole (μM) | | | | | | | | |
|---|---|---|---|---|---|---|---|---|---|---|
| | | 0 | 0.22 | 0.44 | 0.87 | 1.7 | 3.5 | 7 | 14 | 28 |
| Atorvastatin (μM) | 0 | −2.5 | −1.5 | −1.3 | 1.7 | 6.8 | 17.0 | 17.4 | 18.1 | 18.6 |
| | 0.28 | −2.4 | −3.8 | 0.7 | 3.7 | 14.5 | 23.1 | 27.4 | 27.7 | 26.7 |
| | 0.56 | 2.9 | 4.1 | 5.6 | 11.8 | 17.1 | 28.7 | 36.7 | 31.2 | 31.4 |
| | 1.1 | 11.0 | 6.9 | 15.4 | 12.5 | 24.9 | 39.2 | 42.8 | 45.0 | 37.9 |
| | 2.2 | 5.7 | 7.3 | 6.8 | 10.8 | 20.5 | 38.2 | 52.5 | 49.9 | 50.0 |
| | 4.5 | 13.9 | 12.5 | 11.3 | 14.2 | 32.6 | 66.7 | 74.7 | 76.1 | 69.0 |
| | 9 | 13.1 | 13.1 | 21.1 | 24.0 | 62.8 | 78.9 | 82.9 | 83.6 | 83.5 |
| | 18 | 34.6 | 34.0 | 41.4 | 61.5 | 78.7 | 89.0 | 89.6 | 89.9 | 89.6 |
| | 36 | 69.0 | 70.2 | 76.6 | 82.7 | 89.2 | 92.1 | 93.0 | 92.9 | 92.9 |

### EXAMPLE 8

### Antiproliferative Activity of Atorvastatin and Itraconazole Against PANC1 Human Pancreatic Carcinoma Cells

The results from a 2-fold dilution series of atorvastatin and itraconazole on PANC1 cell growth are shown in Table 8. In the present assay, atorvastatin exhibits maximal inhibition of 37.5% at 36 μM. In the absence of itraconazole, 9 μM atorvastatin exhibits 6.8% inhibition, while in the presence of 7 μM itraconazole, the same amount of atovastatin exhibits 36% inhibition, nearly restoring maximal inhibition of proliferation.

TABLE 8
Percent inhibition of Alamar Blue Metabolism in PANC1 cells

| | | Itraconazole (μM) | | | | | | | | |
|---|---|---|---|---|---|---|---|---|---|---|
| | | 0 | 0.22 | 0.44 | 0.87 | 1.7 | 3.5 | 7 | 14 | 28 |
| Atorvastatin (μM) | 0 | −0.6 | −3.6 | 3.0 | 9.8 | 5.5 | 0.2 | −0.6 | −4.4 | −2.5 |
| | 0.28 | −1.6 | 4.9 | 2.1 | 6.6 | 5.9 | 3.8 | 10.3 | 3.5 | 7.0 |
| | 0.56 | 11.3 | 7.7 | 9.8 | 12.3 | 17.2 | 9.0 | 13.0 | 7.3 | 4.0 |
| | 1.1 | 12.2 | 22.3 | 27.1 | 12.0 | 15.3 | 20.9 | 24.8 | 11.8 | 14.9 |
| | 2.2 | 3.4 | 4.3 | 7.4 | 8.6 | 9.0 | 5.3 | 10.2 | 13.6 | 13.6 |
| | 4.5 | 0.5 | 9.4 | 6.3 | 11.3 | 9.5 | 16.0 | 22.4 | 16.5 | 17.2 |
| | 9 | 6.8 | 8.2 | 18.4 | 21.8 | 27.4 | 35.8 | 36.0 | 43.9 | 41.7 |
| | 18 | 15.4 | 19.3 | 22.4 | 28.8 | 31.9 | 45.6 | 43.4 | 43.4 | 51.3 |
| | 36 | 37.5 | 35.6 | 45.2 | 48.7 | 56.7 | 57.5 | 52.8 | 56.6 | 56.9 |

### EXAMPLE 9

### Antiproliferative Activity of Atorvastatin and Itraconazole Against SKMEL-28 Human Melanoma Cells

The results from a 2-fold dilution series of atorvastatin and itraconazole combination on SKMEL-28 cell growth are shown in Table 9. In the present assay, atorvastatin shows maximal inhibition of 45.9% at 36 μM. In the absence of intraconazole, 9 μM simvastatin exhibits 24.0% inhibition, while in the presence of 7 μM itraconazole, inhibition of cell proliferation to 61.8%, exceeding maximal inhibition by atorvastatin alone.

TABLE 9
Percent inhibition of Alamar Blue Metabolism in SKMEL-28 cells

| | | Itraconazole (μM) | | | | | | | | |
|---|---|---|---|---|---|---|---|---|---|---|
| | | 0 | 0.22 | 0.44 | 0.87 | 1.7 | 3.5 | 7 | 14 | 28 |
| Atorvastatin (μM) | 0 | 0.7 | 3.6 | 3.0 | 1.4 | 2.6 | 7.5 | 11.2 | 13.0 | 13.5 |
| | 0.28 | 18.7 | 10.2 | 10.1 | 21.7 | 12.9 | 15.0 | 26.0 | 24.8 | 13.7 |
| | 0.56 | 14.4 | 18.4 | 27.2 | 33.7 | 45.9 | 49.9 | 41.6 | 30.1 | 18.0 |
| | 1.1 | 38.6 | 37.3 | 23.6 | 33.3 | 50.6 | 37.5 | 51.8 | 51.5 | 41.2 |
| | 2.2 | 5.0 | 5.4 | 6.6 | 9.6 | 17.7 | 33.5 | 48.7 | 42.7 | 42.3 |
| | 4.5 | 10.5 | 11.7 | 15.0 | 18.8 | 36.6 | 57.9 | 57.2 | 57.7 | 58.5 |
| | 9 | 24.0 | 21.4 | 26.0 | 41.5 | 56.5 | 60.8 | 61.8 | 59.9 | 61.4 |
| | 18 | 34.1 | 35.1 | 36.2 | 40.9 | 51.2 | 63.1 | 59.2 | 61.0 | 59.4 |
| | 36 | 45.9 | 43.9 | 45.8 | 43.3 | 53.9 | 64.1 | 66.1 | 63.8 | 65.9 |

### EXAMPLE 10

### Antiproliferative Activity of Fluvastatin and Itraconazole Against DU145 Human Prostate Cancer Cells

The results from a 2-fold dilution series of a fluvastatin and itraconazole alone or in combination DU145 cell growth are shown in Table 10. In the present assay, fluvastatin exhibits maximal inhibition of 58.3% at 49 μM. In the absence 12 μM fluvastatin exhibits 15.5% inhibition, and in the presence of 14 μM itraconazole, the efficacy of fluvastatin is enhanced, exhibiting 40.3% inhibition.

TABLE 10
Percent inhibition of Alamar Blue Metabolism in DU145 cells

| | | Itraconazole (μM) | | | | | | | | |
|---|---|---|---|---|---|---|---|---|---|---|
| | | 0 | 0.22 | 0.44 | 0.87 | 1.7 | 3.5 | 7 | 14 | 28 |
| Fluvastatin (μM) | 0 | −1.0 | 2.0 | −0.6 | 2.1 | 0.6 | −3.3 | −7.4 | −5.6 | −9.0 |
| | 0.38 | 2.8 | −2.1 | 1.2 | −1.0 | 1.4 | −0.9 | 2.2 | −0.2 | −2.9 |
| | 0.76 | −6.1 | 1.9 | 0.8 | 3.9 | −5.2 | −0.3 | −1.5 | 0.7 | −1.9 |
| | 1.5 | 1.2 | −3.8 | 4.4 | 8.1 | 3.7 | 6.3 | 4.7 | 1.7 | 8.8 |
| | 3 | −1.1 | 0.5 | 11.4 | 5.0 | 1.6 | 5.6 | 6.9 | 5.9 | 3.6 |
| | 6.1 | 1.8 | 3.3 | 9.8 | 7.8 | 6.9 | 13.2 | 24.2 | 20.9 | 20.2 |
| | 12 | 15.5 | 18.2 | 26.1 | 22.1 | 25.4 | 33.6 | 41.8 | 40.3 | 42.9 |
| | 24 | 38.9 | 42.5 | 43.1 | 42.4 | 42.2 | 50.6 | 49.7 | 48.6 | 48.0 |
| | 49 | 58.3 | 61.3 | 59.7 | 60.0 | 58.4 | 54.3 | 55.9 | 57.2 | 57.7 |

### EXAMPLE 11

### Antiproliferative Activity of Fluvastatin and Itraconazole Against HCT116 Human Colorectal Carcinoma

The results from a 2-fold dilution series of a fluvastatin and itraconazole combination on HCT116 cell growth are shown in Table 11. In the present assay, fluvastatin exhibits maximal inhibition of 93.1% at 49 μM. 6.1 μM fluvastatin, in the presence of 7 μM itraconazole, exhibits 91.9% inhibition, providing a dose sparing effect that allows nearly maximal inhibition with an 8 fold reduction in the concentration of fluvastatin.

TABLE 11
Percent inhibition of Alamar Blue Metabolism in HCT116 cells

| | | Itraconazole (μM) | | | | | | | | |
|---|---|---|---|---|---|---|---|---|---|---|
| | | 0 | 0.22 | 0.44 | 0.87 | 1.7 | 3.5 | 7 | 14 | 28 |
| Fluvastatin (μM) | 0 | −2.5 | −1.2 | −0.6 | 2.2 | 9.3 | 16.7 | 18.9 | 20.2 | 20.4 |
| | 0.38 | 6.3 | 6.4 | 4.4 | 6.8 | 16.5 | 35.0 | 41.7 | 41.1 | 40.3 |
| | 0.76 | 16.4 | 16.5 | 17.2 | 20.3 | 38.5 | 57.6 | 69.9 | 65.0 | 63.5 |
| | 1.5 | 32.5 | 40.5 | 30.7 | 36.9 | 59.1 | 82.0 | 85.8 | 85.6 | 78.9 |
| | 3 | 38.5 | 40.2 | 47.6 | 55.9 | 79.0 | 87.5 | 88.3 | 88.7 | 88.1 |
| | 6.1 | 57.7 | 66.1 | 68.3 | 81.5 | 86.4 | 91.4 | 91.9 | 92.0 | 91.2 |
| | 12 | 78.3 | 77.7 | 83.4 | 86.5 | 90.2 | 92.5 | 93.2 | 93.0 | 93.2 |
| | 24 | 86.7 | 87.9 | 89.2 | 90.8 | 92.2 | 93.9 | 94.1 | 94.2 | 94.0 |
| | 49 | 93.1 | 93.1 | 93.4 | 93.6 | 93.9 | 94.6 | 94.4 | 94.6 | 94.5 |

### EXAMPLE 12

### Antiproliferative Activity of Fluvastatin and Itraconazole Against PANC1 Human Pancreatic Carcinoma

The results from a 2-fold dilution series of an fluvastatin and itraconazole combination on PANC1 cell growth are shown in Table 12. In the present assay, fluvastatin exhibits maximal inhibition of 59.9% at 49 μM. However, the combination of 7 μM itraconazole with 12 μM fluvastatin exhibits 56.8% inhibition, enabling the use of one fourth the amount of fluvastatin and still achieve nearly maximal inhibition.

TABLE 12
Percent inhibition of Alamar Blue Metabolism in PANC1 cells

| | | Itraconazole (μM) | | | | | | | | |
|---|---|---|---|---|---|---|---|---|---|---|
| | | 0 | 0.22 | 0.44 | 0.87 | 1.7 | 3.5 | 7 | 14 | 28 |
| Fluvastatin (μM) | 0 | −3.3 | −1.0 | 5.4 | 4.9 | 9.3 | 2.1 | −1.9 | −8.1 | −3.7 |
| | 0.38 | −3.2 | 2.8 | 1.0 | 3.0 | −2.5 | 6.0 | 10.8 | 9.0 | 7.0 |
| | 0.76 | 11.3 | 9.8 | 20.6 | 16.5 | 16.7 | 21.3 | 35.6 | 27.1 | 30.4 |
| | 1.5 | 13.9 | 18.3 | 20.3 | 26.2 | 30.8 | 49.9 | 52.6 | 42.7 | 45.7 |
| | 3 | 23.1 | 24.4 | 29.5 | 28.1 | 40.1 | 43.1 | 49.5 | 44.8 | 52.6 |
| | 6.1 | 33.0 | 38.7 | 36.2 | 44.4 | 47.8 | 47.8 | 50.6 | 46.4 | 50.5 |
| | 12 | 45.3 | 44.5 | 51.5 | 56.1 | 57.8 | 55.5 | 56.8 | 57.1 | 55.4 |
| | 24 | 51.3 | 56.0 | 53.2 | 57.1 | 54.5 | 55.6 | 55.5 | 57.5 | 57.2 |
| | 49 | 59.9 | 57.1 | 59.7 | 58.2 | 55.3 | 53.4 | 59.0 | 52.4 | 53.5 |

### EXAMPLE 13

### Antiproliferative Activity of Fluvastatin and Itraconazole Against SKMEL-28 Human Melanoma Cells

The results from a 2-fold dilution series of fluvastatin and itraconazole combination on SKMEL-28 cell growth are shown in Table 13. In the present assay, fluvastatin exhibits maximal inhibition of 27.8% at 49 μM. In the presence of 7 μM itraconazole and 3 μM fluvastatin, a 16 fold reduction in the concentration of fluvastatin, the combination exhibits 59.5% inhibition more than doubling the maximal inhibition of fluvastatin alone.

TABLE 13
Percent inhibition of Alamar Blue Metabolism in SKMEL-28 cells

| | | Itraconazole (μM) | | | | | | | | |
|---|---|---|---|---|---|---|---|---|---|---|
| | | 0 | 0.22 | 0.44 | 0.87 | 1.7 | 3.5 | 7 | 14 | 28 |
| Fluvastatin (μM) | 0 | −0.2 | 0.4 | 0.9 | 2.8 | 9.8 | 7.5 | 11.3 | 13.3 | 16.3 |
| | 0.38 | 9.5 | 1.8 | 5.1 | 3.3 | 10.7 | 18.7 | 29.9 | 30.2 | 21.7 |
| | 0.76 | 9.3 | 15.2 | 12.5 | 19.0 | 22.9 | 37.8 | 45.1 | 44.4 | 50.7 |
| | 1.5 | 22.9 | 29.7 | 25.9 | 35.4 | 33.4 | 57.6 | 57.6 | 55.6 | 56.0 |
| | 3 | 18.2 | 20.3 | 25.0 | 31.2 | 51.1 | 57.1 | 59.5 | 56.8 | 57.8 |
| | 6.1 | 26.5 | 27.4 | 30.1 | 35.4 | 49.6 | 56.1 | 56.5 | 56.2 | 54.8 |
| | 12 | 30.7 | 30.0 | 31.5 | 34.8 | 43.5 | 49.4 | 50.2 | 50.3 | 50.8 |
| | 24 | 30.8 | 30.1 | 30.8 | 32.6 | 36.7 | 46.1 | 47.3 | 49.1 | 47.0 |
| | 49 | 27.8 | 26.6 | 26.8 | 26.4 | 28.9 | 34.2 | 41.0 | 40.6 | 42.3 |

### EXAMPLE 14

### Antiproliferative Activity of Lovastatin and Itraconazole Against HCT116 Human Colorectal Carcinoma Cells

The results from a 2-fold dilution series of lovastatin and itraconazole, either alone or in combination, on HCT116 cell growth are shown in Table 15. In the present assay, lovastatin exhibits maximal inhibition of 55.0% at 48 μM. In the presence of 3.5 μM itraconazole, 6 μM lovastatin exhibits 83.4% inhibition. Thus, with an 8-fold reduction in the concentration of lovastatin, the combination exceeds the maximal inhibition exhibited by lovastatin alone.

TABLE 14
Percent inhibition of Alamar Blue Metabolism in HCT116 cells

| | | Itraconazole (μM) | | | | | | | | |
|---|---|---|---|---|---|---|---|---|---|---|
| | | 0 | 0.22 | 0.44 | 0.87 | 1.7 | 3.5 | 7 | 14 | 28 |
| Lovastatin (μM) | 0 | −2.7 | −0.1 | −0.8 | 5.1 | 6.8 | 14.2 | 17.8 | 19.5 | 20.7 |
| | 0.38 | −0.4 | −1.6 | 4.9 | 3.1 | 12.7 | 23.6 | 27.7 | 31.8 | 31.6 |
| | 0.75 | 1.3 | 7.3 | 1.9 | 6.2 | 12.8 | 26.0 | 41.0 | 40.2 | 38.7 |
| | 1.5 | 16.7 | 8.5 | 14.4 | 13.2 | 27.0 | 46.5 | 65.9 | 66.9 | 61.8 |
| | 3 | 24.4 | 20.2 | 23.5 | 29.7 | 45.1 | 71.5 | 79.8 | 79.7 | 80.2 |
| | 6 | 36.6 | 45.3 | 40.0 | 47.4 | 65.5 | 83.4 | 86.2 | 88.2 | 85.7 |
| | 12 | 44.0 | 40.1 | 48.8 | 64.1 | 74.8 | 86.8 | 89.6 | 88.9 | 89.3 |
| | 24 | 46.9 | 49.9 | 57.6 | 63.9 | 71.6 | 86.2 | 89.4 | 88.9 | 88.8 |
| | 48 | 55.0 | 53.3 | 54.7 | 58.9 | 64.5 | 78.4 | 87.6 | 87.0 | 87.2 |

### EXAMPLE 15

### Antiproliferative Activity of Lovastatin and Itraconazole Against HCT116 Human Colorectal Carcinoma Cells

The results from a 2-fold dilution series of a lovastatin sodium (Calbiochem) and itraconazole combination on HCT116 cell growth are shown in Table 15. In the present assay, lovastatin exhibits maximal inhibition of 58.9% at 22 μM. In the presence of 7 μm itraconazole, 2.8 μM lovastatin exhibits 82.0% inhibition; with an 8-fold reduction in the concentration of lovastatin, the combination exceeds the maximal inhibition exhibited lovastatin alone.

TABLE 15
Percent inhibition of Alamar Blue Metabolism in HCT116 cells

| | | Itraconazole (μM) | | | | | | | | |
|---|---|---|---|---|---|---|---|---|---|---|
| | | 0 | 0.22 | 0.44 | 0.87 | 1.7 | 3.5 | 7 | 14 | 28 |
| Lovastatin (μM) | 0 | −9.3 | −13.4 | −13.7 | −10.9 | −5.3 | 2.1 | 18.0 | 15.1 | 9.1 |
| | 0.18 | −12.6 | −13.5 | −11.4 | −12.5 | −3.9 | 17.0 | 16.3 | 29.6 | 22.7 |
| | 0.35 | −6.2 | −8.9 | −10.3 | −6.8 | 1.3 | 19.4 | 30.5 | 42.5 | 34.1 |
| | 0.7 | −1.7 | −10.7 | −3.5 | 6.4 | 7.2 | 30.2 | 58.7 | 46.4 | 62.0 |
| | 1.4 | 9.4 | 6.4 | 21.1 | 7.9 | 35.9 | 42.4 | 68.3 | 64.4 | 69.8 |
| | 2.8 | 16.3 | 4.0 | 23.2 | 27.1 | 42.5 | 73.6 | 82.0 | 81.2 | 81.8 |
| | 5.6 | 26.7 | 38.3 | 28.4 | 37.4 | 51.5 | 77.9 | 88.5 | 86.6 | 88.9 |
| | 11 | 43.6 | 41.1 | 54.9 | 64.5 | 77.8 | 90.7 | 91.1 | 90.9 | 91.5 |
| | 22 | 58.9 | 53.8 | 65.3 | 71.4 | 84.7 | 91.2 | 92.9 | 92.6 | 92.9 |

### EXAMPLE 16

### Antiproliferative Activity of Cerivastatin and Itraconazole Against HCT116 Human Colorectal Carcinoma Cells

The results from a 4-fold dilution series of a cerivastatin and itraconazole combination on HCT116 cell growth are shown in Table 16. In the present assay, cerivastatin exhibits maximal inhibition of 85.5% at 22 μM. In the presence of 1.7 μM itraconazole, 1.4 μM cerivastatin exhibits 84.1% inhibition. Thus, an 8-fold reduction in the concentration of cerivastatin showed nearly maximal inhibition in combination with itraconazole.

TABLE 16
Percent inhibition of Alamar Blue Metabolism in HCT116 cells

| | | Cerivastatin (μM) | | | | | |
|---|---|---|---|---|---|---|---|
| | | 0 | 0.085 | 0.34 | 1.4 | 5.4 | 22 |
| Itraconazole (μM) | 0 | −6.73 | 3.09 | 12.8 | 66.4 | 84.5 | 85.5 |
| | 0.11 | −5.7 | −3.37 | 10.1 | 68.1 | 84 | 85.9 |
| | 0.44 | −6.67 | −1.88 | 21.8 | 76.3 | 86.5 | 87.7 |
| | 1.7 | −3.84 | 16.3 | 73.2 | 84.1 | 88.4 | 87.2 |
| | 7 | 24.3 | 55.8 | 80.1 | 89 | 89.7 | 88.8 |
| | 28 | 27.7 | 60.1 | 81.9 | 89.7 | 89.9 | 87.9 |

### EXAMPLE 17

### Antiproliferative Activity of Atorvastatin and Clotrimazole Against HCT116 Human Colorectal Carcinoma Cells

The results from a 2-fold dilution series of atorvastatin and clotrimazole in combination on HCT116 cell growth are shown in Table 17. In the present assay, atorvastatin exhibits maximal inhibition of 89.3% at 36 μM. In the presence of 7.3 μM clotrimazole and 18 μM atorvastatin, the combination exhibits 82.7% inhibition.

TABLE 17
Percent inhibition of Alamar Blue Metabolism in HCT116 cells

| | | Atorvastatin (μM) | | | | | | | | |
|---|---|---|---|---|---|---|---|---|---|---|
| | | 0.0 | 0.3 | 0.6 | 1.1 | 2.2 | 4.5 | 9.0 | 18.0 | 36.0 |
| Clotrimazole (μM) | 0.0 | 11.2 | 8.1 | 19.3 | 4.5 | 21.3 | 37.4 | 56.8 | 59.8 | 89.3 |
| | 0.5 | 25.9 | 31.3 | 23.4 | 16.9 | 53.6 | 49.0 | 50.8 | 79.3 | 85.2 |
| | 0.9 | 35.8 | 63.7 | 54.4 | 64.1 | 63.7 | 80.0 | 81.4 | 89.8 | 91.1 |
| | 1.8 | 54.3 | 61.6 | 71.9 | 74.8 | 77.2 | 75.6 | 85.7 | 84.8 | 92.6 |
| | 3.6 | 13.2 | 8.0 | 8.2 | 21.1 | 30.0 | 48.3 | 56.5 | 66.2 | 89.7 |
| | 7.3 | 19.0 | 13.3 | 27.6 | 22.8 | 47.1 | 54.6 | 55.7 | 82.7 | 93.0 |
| | 15.0 | 35.6 | 25.5 | 33.3 | 37.7 | 57.7 | 42.9 | 74.6 | 80.5 | 93.5 |
| | 29.0 | 51.5 | 62.2 | 48.5 | 68.4 | 63.7 | 84.8 | 82.4 | 91.8 | 90.4 |
| | 58.0 | 95.0 | 94.8 | 95.3 | 94.3 | 95.1 | 94.5 | 92.8 | 94.9 | 92.8 |

### EXAMPLE 18

### Antiproliferative Activity of Atorvastatin and Econazole Against HCT116 Human Colorectal Carcinoma Cells

The results from a 2-fold dilution series of atorvastatin and econazole in combination on HCT116 cell growth are shown in Table 18. In the present assay, econazole exhibits maximal inhibition of 83.1% at 36 μM. In the presence of 11.0 μM econazole and 18 μM atorvastatin, the combination exhibits 80.9% inhibition.

TABLE 18
Percent inhibition of Alamar Blue Metabolism in HCT116 cells

| | | Atorvastatin (μM) | | | | | | | | |
|---|---|---|---|---|---|---|---|---|---|---|
| | | 0.0 | 0.3 | 0.6 | 1.1 | 2.2 | 4.5 | 9.0 | 18.0 | 36.0 |
| Econazole (μM) | 0.0 | 1.2 | 2.7 | 2.5 | 3.4 | 12.6 | 19.7 | 32.8 | 53.0 | 83.1 |
| | 0.4 | 0.7 | 1.9 | 5.3 | 8.0 | 16.4 | 22.9 | 33.8 | 56.4 | 82.4 |
| | 0.7 | −1.7 | 1.7 | 4.1 | 7.5 | 15.3 | 24.4 | 35.4 | 59.7 | 84.5 |
| | 1.4 | 1.8 | 2.0 | 3.4 | 5.2 | 14.2 | 22.1 | 39.7 | 59.3 | 85.5 |
| | 2.8 | 1.6 | 1.6 | 3.6 | 7.0 | 14.1 | 23.6 | 47.0 | 60.9 | 85.4 |
| | 5.6 | 6.5 | 9.7 | 9.4 | 14.3 | 20.1 | 30.7 | 43.3 | 65.8 | 89.4 |
| | 11.0 | 11.1 | 26.7 | 12.9 | 21.0 | 32.4 | 46.4 | 65.0 | 80.9 | 93.0 |
| | 22.0 | 56.8 | 68.3 | 63.1 | 84.1 | 84.5 | 88.2 | 91.7 | 93.9 | 94.3 |
| | 45.0 | 96.3 | 96.4 | 96.3 | 96.4 | 96.4 | 96.3 | 96.4 | 96.3 | 96.3 |

### EXAMPLE 19

### Antiproliferative Activity of Atorvastatin and Ketoconazole Against HCT116 Human Colorectal Carcinoma Cells

The results from a 2-fold dilution series of atorvastatin and ketoconazole in combination on HCT116 cell growth are shown in Table 19. In the present assay, atorvastatin exhibits maximal inhibition of 73.7% at 36 μM. In the presence of 4.7 μM ketoconazole and 18 μM atorvastatin, the combination exhibits 68.3% inhibition.

TABLE 19
Percent inhibition of Alamar Blue Metabolism in HCT116 cells

| | | Atorvastatin(μM) | | | | | | | | |
|---|---|---|---|---|---|---|---|---|---|---|
| | | 0 | 0.28 | 0.56 | 1.1 | 2.2 | 4.5 | 9 | 18 | 36 |
| Ketoconazole (μM) | 0 | −7.3 | −4.3 | 0.8 | 4.7 | 18.8 | 13.3 | 20.4 | 42.7 | 73.7 |
| | 0.29 | 3.9 | 11.9 | 6.7 | 6.5 | 8.9 | 32.5 | 39.0 | 61.8 | 77.7 |
| | 0.59 | 4.8 | −4.1 | 5.7 | 4.5 | 12.5 | 20.4 | 42.5 | 63.3 | 83.0 |
| | 1.2 | 19.0 | 6.4 | 0.7 | 14.4 | 8.9 | 38.3 | 41.0 | 69.6 | 84.0 |
| | 2.4 | 5.3 | 11.3 | 7.0 | 26.9 | 32.2 | 47.8 | 58.9 | 64.4 | 80.5 |
| | 4.7 | 3.1 | 21.2 | 25.6 | 38.3 | 31.4 | 44.1 | 57.1 | 68.3 | 85.9 |
| | 9.4 | 10.0 | 18.3 | 12.5 | 15.0 | 20.1 | 21.4 | 35.3 | 57.9 | 88.2 |
| | 19 | 23.8 | 37.0 | 20.7 | 31.0 | 47.5 | 50.5 | 35.9 | 62.2 | 84.1 |
| | 38 | 40.0 | 46.4 | 53.0 | 42.0 | 54.5 | 59.7 | 65.1 | 74.8 | 90.4 |

### EXAMPLE 20

### Antiproliferative Activity of Lovastatin and Econazole Against HCT116 Human Colorectal Carcinoma Cells

The results from a 2-fold dilution series of lovastatin and econazole in combination on HCT116 cell growth are shown in Table 20. In the present assay, lovastatin exhibits maximal inhibition of 95.6% at 36 μM. In the presence of 12.0 μM econazole and 18 μM lovastatin, the combination exhibits 84.5% inhibition.

TABLE 20
Percent inhibition of Alamar Blue Metabolism in HCT116 cells

| | | Lovastatin (μM) | | | | | | | | |
|---|---|---|---|---|---|---|---|---|---|---|
| | | 0.0 | 0.3 | 0.6 | 1.1 | 2.2 | 4.5 | 9.0 | 18.0 | 36.0 |
| Econazole (μM) | 0.0 | 12.0 | 10.5 | 5.4 | 2.6 | 1.9 | 20.2 | 27.3 | 41.3 | 95.6 |
| | 0.4 | 0.6 | 13.9 | 29.5 | 30.0 | 2.4 | 33.7 | 35.4 | 63.7 | 95.7 |
| | 0.8 | 8.8 | 7.8 | 20.7 | 7.7 | 14.0 | 27.8 | 53.7 | 44.9 | 95.6 |
| | 1.5 | 14.9 | 12.8 | 15.0 | 29.5 | 30.8 | 54.2 | 51.4 | 64.4 | 95.6 |
| | 3.0 | 23.9 | 22.1 | 24.5 | 27.6 | 27.2 | 32.2 | 44.6 | 65.2 | 95.8 |
| | 6.0 | 36.7 | 40.3 | 38.0 | 46.3 | 44.8 | 44.3 | 50.6 | 78.5 | 96.0 |
| | 12.0 | 57.2 | 58.8 | 60.6 | 61.8 | 59.8 | 59.6 | 66.1 | 84.5 | 96.1 |
| | 24.0 | 78.6 | 79.3 | 75.2 | 76.6 | 77.3 | 78.1 | 81.5 | 91.0 | 96.2 |
| | 48.0 | 96.1 | 96.1 | 95.9 | 96.1 | 95.8 | 96.1 | 95.9 | 96.0 | 96.1 |

### EXAMPLE 21

### Antiproliferative Activity of Atorvastatin and Terconazole Against HCT116 Human Colorectal Carcinoma Cells

The results from a 2-fold dilution series of atorvastatin and terconazole in combination on HCT116 cell growth are shown in Table 21. In the present assay, atorvastatin exhibits maximal inhibition of 66.4% at 36 μM. In the presence of 19.0 μM terconazole and 18 μM atorvastatin, the combination exhibits 78.4% inhibition.

TABLE 21
Percent inhibition of Alamar Blue Metabolism in HCT116 cells

| | | Atorvastatin (μM) | | | | | | | | |
|---|---|---|---|---|---|---|---|---|---|---|
| | | 0 | 0.28 | 0.56 | 1.1 | 2.2 | 4.5 | 9 | 18 | 36 |
| Terconazole | 0 | −1.9 | 0.3 | −0.1 | 3.3 | 8.5 | 12.9 | 21.0 | 40.2 | 66.4 |
| (μM) | 0.29 | −1.8 | −1.9 | 1.1 | 1.3 | 8.3 | 20.9 | 22.8 | 45.6 | 64.3 |
| | 0.59 | 1.6 | 0.7 | 1.4 | 11.8 | 10.7 | 24.7 | 26.6 | 46.7 | 75.6 |
| | 1.2 | 1.7 | 1.2 | 4.3 | 6.4 | 14.3 | 18.3 | 38.5 | 52.4 | 75.6 |
| | 2.3 | 5.0 | 10.0 | 4.5 | 19.2 | 12.6 | 21.9 | 46.5 | 54.7 | 80.0 |
| | 4.7 | 1.1 | 1.5 | 3.6 | 8.8 | 16.5 | 22.7 | 31.0 | 44.1 | 73.2 |
| | 9.4 | 8.2 | 8.8 | 14.9 | 14.1 | 15.5 | 18.5 | 18.5 | 31.8 | 81.4 |
| | 19 | 38.4 | 43.8 | 41.5 | 57.4 | 48.5 | 57.4 | 61.4 | 78.4 | 93.1 |
| | 38 | 94.8 | 94.5 | 95.7 | 95.5 | 95.8 | 95.2 | 95.9 | 95.8 | 95.9 |

### EXAMPLE 22

### Antiproliferative Activity of Lovastatin and Tioconazole Against HCT116 Human Colorectal Carcinoma Cells

The results from a 2-fold dilution series of lovastatin and tioconazole in combination on HCT116 cell growth are shown in Table 22. In the present assay, lovastatin exhibits maximal inhibition of 61.9% at 24 μM. In the presence of 9.0 μM tioconazole and 6.0 μM lovastatin, the combination exhibits 70.1% inhibition.

TABLE 22
Percent inhibition of Alamar Blue Metabolism in HCT116 cells

| | | Tioconazole (μM) | | | | | | | | |
|---|---|---|---|---|---|---|---|---|---|---|
| | | 0.0 | 0.3 | 0.6 | 1.1 | 2.2 | 4.5 | 9.0 | 18.0 | 36.0 |
| Lovastatin (μM) | 0.0 | 7.4 | 8.6 | 3.7 | 8.5 | 9.8 | 6.8 | 19.8 | 26.8 | 41.3 |
| | 0.2 | 9.8 | 11.4 | 5.7 | 7.0 | 11.2 | 6.3 | 11.8 | 13.8 | 37.7 |
| | 0.4 | 23.1 | 21.7 | 16.0 | 15.8 | 16.9 | 13.0 | 23.2 | 51.0 | 45.8 |
| | 0.8 | 12.3 | 17.3 | 17.2 | 18.1 | 18.2 | 21.5 | 52.7 | 47.9 | 58.0 |
| | 1.5 | 32.2 | 26.7 | 28.5 | 23.5 | 34.5 | 32.0 | 47.6 | 57.9 | 75.4 |
| | 3.0 | 29.9 | 37.0 | 34.6 | 33.3 | 42.2 | 48.4 | 48.9 | 76.0 | 85.5 |
| | 6.0 | 46.1 | 41.8 | 40.0 | 41.0 | 53.6 | 51.8 | 70.1 | 79.1 | 90.6 |
| | 12.0 | 50.0 | 52.1 | 49.6 | 52.3 | 48.6 | 60.1 | 69.2 | 86.4 | 93.1 |
| | 24.0 | 61.9 | 63.6 | 62.7 | 62.2 | 65.0 | 69.6 | 76.5 | 90.6 | 94.2 |

Materials and Methods

The foregoing results were obtained with the following materials and methods.

Tumor Cell Culture

Human pancreatic cancer PANC1 cells (ATCC# CRL-1469), human colorectal carcinoma HCT116 cells (ATCC# CCL-247), non-small cells lung carcinoma A549 cells (ATCC# CCL-185), human prostate cancer DU145 cells (ATCC# HTB-81), and human melanoma SKMEL-28 cells (ATCC# HTB-72) were grown at 37±0.5° C. and 5% CO₂ in RPMI 1640 medium supplemented with 10% FBS, 2 mM glutamine, 1% penicillin, and 1% streptomycin.

Test Compounds

Clotrimazole, econazole, ketoconazole, lovastatin, and simvastatin were obtained from Sigma Chemical Co. (St. Louis, Mo.). Atorvastatin, itraconazole, and terconazole were obtained from Intrachem (Paramus, N.J.). Cerivastatin and fluvastatin were obtained from Sequoia Research Products (Oxford, United Kingdom). Lovastatin sodium and simvastatin sodium were obtained from Calbiochem (San Diego, Calif.). Stock solutions (1000×) of each compound were prepared in DMSO and stored at −20° C. Master stock plates of 2-fold or 4-fold serial dilutions of individual compounds were prepared in 384-well plates. Combination matrices of test compounds were generated from these master stock plates by dilution into growth media described above. The final concentration of test compounds in the combination matrices was 10× greater than used in the assay. The combination matrices were used immediately and discarded.

Anti-Proliferation Assay

The anti-proliferation assays were performed in 384-well plates. The tumor cells were liberated from the culture flask using a solution of 0.25% trypsin. Cells were diluted in culture media such that 3,000 SKMEL-28 cells, or 1,500 cells for all the other cell lines, were delivered in 35 μl of media into each assay well. Assay plates were incubated for 16-24 hours 37° C.±0.5° C. with 5% CO₂. After incubation, 4.5 μl of 10× stock solutions from the combination matrices were added to 40 μl of culture media. Assay plates were further incubated for 72 hours at 37° C.±0.5° C. with 5% CO₂. Forty microliters of 10.5% Alamar Blue warmed to 37° C.±0.5° C. was added to each assay well following the incubation period. Alamar Blue metabolism was quantified by the amount of fluorescence intensity 3.5-5.0 hours after addition. Quantification, using an LJL Analyst AD reader (LJL Biosystems), was taken in the middle of the well with high attenuation, a 100 msec read time, an excitation filter at 530 nm, and an emission filter at 575 nm. For some experiments, quantification was performed using a Wallac Victor² reader. Measurements were taken at the top of the well with stabilized energy lamp control; a 100 msec read time, an excitation filter at 530 nm, and an emission filter at 590 nm. No significant differences between plate readers were measured.

The percent inhibition (% I) for each well was calculated using the following formula:
% I=[(avg. untreated wells−treated well)/(avg. untreated wells)]×100
The average untreated well value (avg. untreated wells) is the arithmetic mean of 40 wells from the same assay plate treated with vehicle alone. Negative inhibition values result from local variations in treated wells as compared to untreated wells.

### Other Embodiments

The anti-proliferative effect demonstrated with the tumor cell lines used herein can be similarly demonstrated using other cancer cell lines, such as NSC lung carcinoma, MCF7 mammary adenocarcinoma, PA-1 ovarian teratocarcinoma, HT29 colorectal adenocarcinoma, H1299 large cell carcinoma, U-2 OS osteogenic sarcoma, U-373 MG glioblastoma, Hep-3B hepatocellular carcinoma, BT-549 mammary carcinoma, T-24 bladder cancer, C-33A cervical carcinoma, HT-3 metastatic cervical carcinoma, SiHa squamous cervical carcinoma, CaSki epidermoid cervical carcinoma, NCI-H292 mucoepidermoid lung carcinoma, NCI-2030, non small cell lung carcinoma, HeLa, epithelial cervical adenocarcinoma, KB epithelial mouth carcinoma, HT1080 epithelial fibrosarcoma, Saos-2 epithelial osteogenic sarcoma, PC3 epithelial prostate adenocarcinoma, SW480 colorectal carcinoma, CCL-228, MS-751 epidermoid cervical carcinoma, LOX IMVI melanoma, MALME-3M melanoma, M14 melanoma, SK-MEL-2 melanoma, SK-MEL-28 melanoma, SK-MEL-5 melanoma, UACC-257 melanoma, and UACC-62 melanoma cell lines. The specificity can be tested by using cells such as NHLF lung fibroblasts, NHDF dermal fibroblasts, HMEC mammary epithelial cells, PrEC prostate epithelial cells, HRE renal epithelial cells, NHBE bronchial epithelial cells, CoSmC Colon smooth muscle cells, CoEC colon endothelial cells, NHEK epidermal keratinocytes, and bone marrow cells as control cells.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in oncology or related fields are intended to be within the scope of the invention.

We claim:

1. A composition comprising an HMG-CoA reductase inhibitor and an azole, wherein the HMG-CoA reductase inhibitor and the azole are present in amounts effective for the treatment of a neoplasm.

2. The composition of claim 1, wherein the HMG-CoA reductase inhibitor is selected from the group consisting of simvastatin, lovastatin, mevastatin, pravastatin, monacolin M, monacolin X, fluvastatin, atorvastatin, cerivastatin, rosuvastatin, fluindostatin, velostatin, compactin, dihydrocompactin, rivastatin, dalvastatin, and pitavastatin.

3. The composition of claim 1 wherein the azole is selected from the group consisting of fluconazole, itraconazole, hydroxyitraconazole, posaconazole, saperconazole, ketoconazole, clotrimazole, terconazole, econazole, tioconazole, oxiconazole, butoconazole, and miconazole.

4. A method of treating a patient who has a neoplasm or is at risk for developing a neoplasm, said method comprising the step of administering to the patient a composition of claim 1.

5. A method of treating a patient who has a neoplasm or is at risk for developing a neoplasm, comprising administering to the patient an HMG-CoA reductase inhibitor and an azole simultaneously or within 28 days of each other, in amounts sufficient to treat said patient.

6. The method of claim 5, wherein the HMG-CoA reductase inhibitor is selected from the group consisting of simvastatin, lovastatin, mevastatin, pravastatin, monacolin M, monacolin X, fluvastatin, atorvastatin, cerivastatin, rosuvastatin, fluindostatin, velostatin, compactin, dihydrocompactin, rivastatin, dalvastatin, and pitavastatin.

7. The method of claim 5 wherein the azole is selected from the group consisting of fluconazole, itraconazole, hydroxyitraconazole, posaconazole, saperconazole, ketoconazole, clotrimazole, terconazole, econazole, tioconazole, oxiconazole, butoconazole, and miconazole.

8. The method of claim 5, wherein the neoplasm is selected from the group consisting of colon cancer, lung cancer, non-small cell carcinoma, ovarian cancer, prostate cancer, and leukemia.

9. The method of claim 5, wherein the HMG-CoA reductase inhibitor and the azole are administered within 14 days of each other.

10. The method of claim 9, wherein the HMG-CoA reductase inhibitor and the azole are administered within 7 days of each other.

11. The method of claim 10, wherein the HMG-CoA reductase inhibitor and the azole are administered within 24 hours of each other.

12. The method of claim 11, wherein the HMG-CoA reductase inhibitor and the azole are administered within 1 hour of each other.

13. The method of claim 12, wherein the HMG-CoA reductase inhibitor and the azole are administered simultaneously.

14. The method of claim 5, wherein the HMG-CoA reductase inhibitor is administered in amount between 0.1 mg and 100 mg per day.

15. The method of claim 14, wherein the HMG-CoA reductase inhibitor is administered in amount between 0.1 mg and 50 mg per day.

16. The method of claim 15 wherein the HMG-CoA reductase inhibitor is administered in amount between 0.1 mg and 5 mg per day.

17. The method of claim 5, wherein the azole is administered in amount between 0.1 mg and 400 mg per day.

18. The method of claim 17, wherein the azole is administered in amount between 0.1 mg and 200 mg per day.

19. The method of claim 18, wherein the azole is administered in amount between 0.1 mg and 100 mg per day.

20. The method of claim 5, wherein the ratio of azole to HMG-CoA reductase inhibitor administered is at least 4 to 1.

21. The method of claim 20, wherein the ratio of azole to HMG-CoA reductase inhibitor administered is at least 20 to 1.

22. The method of claim 5, wherein a low dose of azole is administered.

23. The method of claim 5, wherein a low dose of HMG-CoA reductase inhibitor is administered.

24. The method of claim 5, further comprising the step of administering to the patient one or more additional cancer treatments selected from the group consisting of surgery, radiation, chemotherapy, immunotherapy, anti-angiogenesis therapy, and gene therapy.

25. A kit, comprising:
(i) a composition, comprising an HMG-CoA reductase inhibitor and an azole; and
(ii) instructions for administering the composition to a patient to treat a neoplasm.

26. A kit, comprising:
(i) an HMG-CoA reductase inhibitor;
(ii) an azole; and
(iii) instructions for administering said HMG-CoA reductase inhibitor and said azole to a patient diagnosed with or at risk of developing a neoplasm.

27. A kit, comprising:
(i) an HMG-CoA reductase inhibitor; and
(ii) instructions for administering said HMG-CoA reductase inhibitor and an azole to a patient diagnosed with or at risk of developing a neoplasm.

28. A kit, comprising:
(i) an azole; and
(iii) instructions for administering said azole and an HMG-CoA reductase inhibitor to a patient diagnosed with or at risk of developing a neoplasm.

29. A method for identifying combinations of compounds useful for treating a neoplasm in a patient in need of such treatment, said method comprising the steps of:
(a) contacting cells in vitro with (i) an HMG-CoA reductase inhibitor or an azole and (ii) a candidate compound; and
(b) determining whether the combination of said HMG-CoA reductase inhibitor or azole and said candidate compound reduces cell growth relative to cells contacted with said HMG-CoA reductase inhibitor or azole but not contacted with said candidate compound or cells contacted with said candidate compound but not with said HMG-CoA reductase inhibitor or azole, wherein a reduction in cell growth identifies said combination as a combination that is useful for treating a patient in need of such treatment.
